(19) United States
(12) Patent Application Publication
(10) Pub. No.: US 2010/0042130 A1
(43) Pub. Date: Feb. 18, 2010

(54) LANCET AND PACKAGING THEREFOR

(75) Inventors: Samuel Mason Curry, Oakland, CA (US); Jonathan M. Wyler, Boston, MA (US)

Correspondence Address: YOUNG BASILE, 3001 WEST BIG BEAVER ROAD, SUITE 624, TROY, MI, 48084, US

(73) Assignee: ABBOTT DIABETES CARE INC., Alameda, CA (US)

(21) Appl. No.: 12/192,323

(22) Filed: Aug. 15, 2008

(51) Int. Cl. A61B 17/32

(52) U.S. Cl. 606/181

(57) ABSTRACT

A lancet device is provided with stackable features to carry a plurality of lancets as a single unit. The lancet device includes a lancet body with a sharp tip at one end for drawing a sample of blood. The lancet body has opposing sides with projections on one of the opposing sides and depressions on the other opposing side. The projections and depressions are configured such that projections on one lancet body securely engages into the depressions of an adjacent lancet body providing a stacked formation. As an alternate, the plurality of lancets can be housed in an elongated member so that the plurality of lancets can be transported as a single unit.

### BACKGROUND

Lancing devices are typically handheld units that permit users to draw blood for testing and diagnostic purposes. These devices include a housing with a piercing aperture, a lancet that contains one or more needles, and a firing mechanism. The firing mechanism typically includes a spring or other biasing means which can be cocked either by insertion of the lancet or by movement of a cocking member. Once the lancing device is cocked, it is placed against the user's skin, often the fingertip. The user can then press a trigger to actuate the firing mechanism, which momentarily drives the sharp tip of the needle through the piercing aperture to puncture the user's skin and draw blood.

A myriad of disposable lancets are available for insertion into the lancing devices. However because of the small size of most lancets, the handling of individual lancets and the installation of the lancets into the lancing device can be difficult. A consideration in the design of lancets is to minimize the difficulty in handling one or more lancets, and to improve the portability of the lancets.

Another consideration in the design of lancing systems is the ease with which a lancet can be inserted into the lancing device while avoiding accidental needle pricks when inserting and removing lancets from the lancing device.

### SUMMARY

A lancet is provided that is stackable or otherwise packaged to carry multiple lancets as a single unit. A lancet is disclosed having a lancet body with opposing ends, a first opposing side and a second opposing side, and a sharp tip protruding from one of the opposing ends, wherein the first and second opposing sides of the lancet body are configured to fittingly engage at least one of a first and second opposing side of an adjacent lancet body.

A lancet package is also disclosed which includes a plurality of lancets each terminating in a sharp tip and an elongated member frangibly attached to each of the plurality of lancets and configured to cover the sharp tip of each of the plurality of lancets in the manner of a cap.

A method is also disclosed for loading a lancing device having a skin contact end with a piercing aperture comprising the steps of providing a lancet package comprised of a plurality of lancets each terminating in a sharp tip and an elongated member frangibly attached to each of the plurality of lancets and configured to cover the sharp tip of each of the plurality lancets in a manner of a cap. The method further includes placing the skin contact end of the lancing device over one of the plurality of lancets such that the lancet engages in a lancet engaging mechanism in the lancing device, applying a forced perpendicular to a central axis of the lancing device sufficient to detach the lancet from the elongated member and arming the lancing device by actuating the lancet engaging mechanism to move the lancet so that the sharp tip is within the lancing device.

### BRIEF DESCRIPTION OF THE DRAWINGS

The various features, advantages and other uses of the present apparatus will become more apparent by referring to the following detailed description and drawing in which:

FIG. 1A is a perspective view of a lancet according to a first embodiment of the invention;

FIG. 1B is a perspective view of an opposing side of the lancet of FIG. 1A;

FIG. 1C is a perspective view of the lancet in FIG. 1A having an alternate shaped projection;

FIG. 2 is a perspective view of a lancet according to a second embodiment of the invention;

FIG. 3 is a front elevational view of a plurality of lancets of FIG. 2 in an engaged formation;

FIG. 4A is a perspective view of a lancet package having an elongate member carrying a plurality of lancets;

FIG. 4B is a perspective view of another embodiment of the lancet package;

FIG. 4C is a perspective view of the lancet package showing a different configuration of the elongate member; and

FIG. 5 is a partial sectional view of a portion of the lancet package.

### DETAILED DESCRIPTION

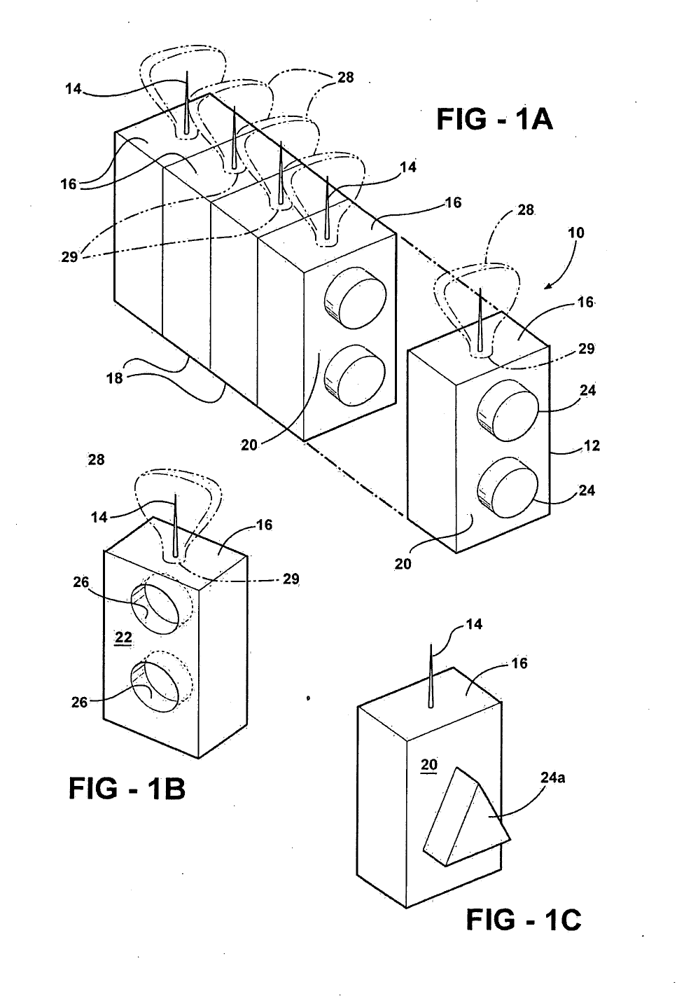

A lancet device is shown in the FIGS. 1-5 which allows for multiple lancets to be carried as a unit to facilitate portability of the lancets. A lancet 10 is shown in FIGS. 1A through 1C according to one embodiment, including a lancet body 12 and a sharp tip 14 for contact with a person's skin to extract a sample of blood for testing. The sharp tip 14 extends from one end of the lancet body 12. In the embodiment shown in FIGS. 1A through 1C, the lancet body 12 has a cubic formation including two opposing ends 16 and 18, a first opposing side 20 and second opposing side 22 defined by surfaces disposed between the two opposing ends 16 and 18. The cubic formation provides a chunky or enlarged device for easy handling of the lancet 10. It is envisioned that the lancet body 12 can have a formation other than the cubic formation shown in FIGS. 1A-1C; and further that the opposing ends 16, 18 and opposing sides 20, 22 of the lancet body 12 can have any surface configuration including a continuous or discontinuous planar surface, a continuous or discontinuous arcuate surface, or a combination having planar and arcuate portions.

The first side 20 of the lancet body 12 includes one or more projections: extending therefrom. By way of example only, a pair of circular projections 24 are shown extending from the first opposing side 20 in FIG. 1B; As shown in FIG. 1C, another example of a projection 24a is shown having a different configuration from the embodiment shown in FIG. 1A. The projection 24a shown in FIG. 1C is only one example of a projection shape configured to prevent misalignment of the lancet body 12 and an adjacent lancet body 12, when the lancets are connected to each other.

The second opposing side 22 of the lancet body 12 is shown in FIG. 1B. The second side 22 has depressions 26 formed therein that are shaped and positioned in order to engage with the projections 24 from an adjacent lancet body 12. The projections 24 and depressions 26 are aligned on the appropriate opposing sides 20, 22 of the lancet body 12 so that when adjacent lancet bodies are engaged, the lancet bodies 12 form a stacked configuration with the sharp tips 14 orientated in the same direction and the opposing ends 16, 18 aligned and forming a planar surface. The stacked lancets, as shown in FIG. 1A, allow a plurality of lancets to be carried or transported as a single unit.

The sharp tip 14 of the lancet body 12 protrudes from one of the opposing ends 16. The sharp tip 14 can be covered with a cap 28 to prevent inadvertent contact with a person's skin until needed. The cap 28 maintains the sharp tip portion in a sterile condition. The cap 28 is configured with a frangible attachment 29 to the lancet body 12 allowing a person to easily remove the cap 28 from the lancet body 12.

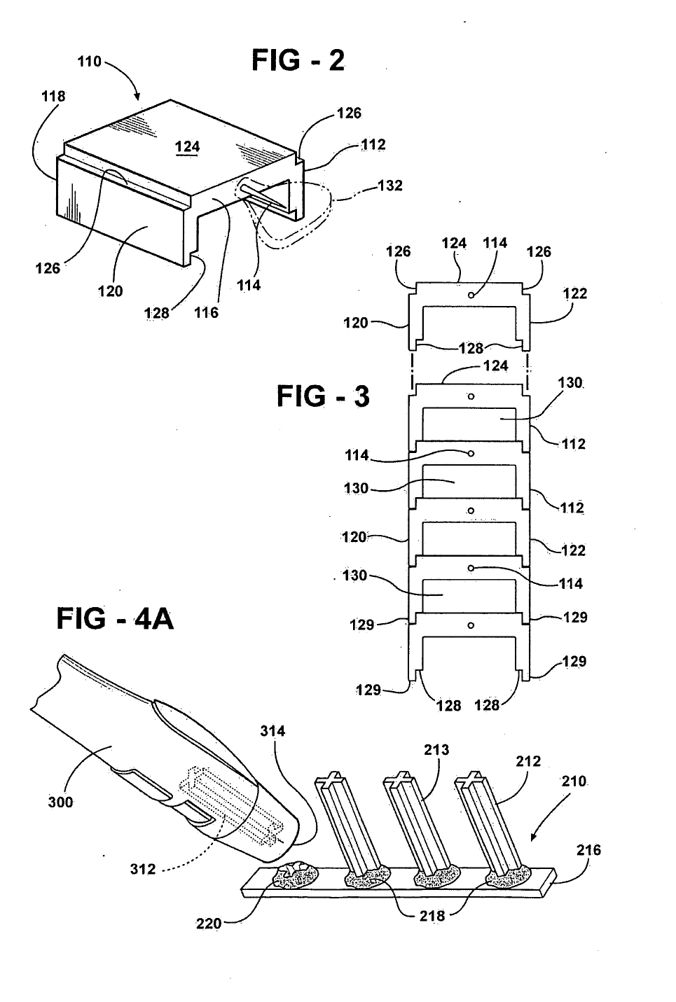

FIGS. 2 and 3 show an alternate embodiment for a stackable lancet. The lancet 110 in FIGS. 2 and 3 include a lancet body 112 and a sharp tip 114 for extracting a sample of blood for testing. The lancet body 112 includes opposing ends 116, 118. The sharp tip portion, 114 protrudes from one of the opposing ends 116. The lancet body 112 further includes a first opposing side 120 and a second opposing side 122. The lancet body 112 also includes a third side 124 disposed between and connected to the first and second opposing sides 120, 122 at corner edges. The third side 124 together with the first and second opposing sides 120, 122 form a U-shaped configuration. In the illustrated embodiment of FIGS. 2 and 3, notches or channels 126 are formed along the corner edge between each of the opposing sides 120, 122 and the third side 124. A corresponding and complementary channel or leg 128 is formed on ah inside edge of the free ends 129 of the first and second opposing sides 120, 122 at a location spaced from the third side 124. The channel or leg 128 of the lancet body 112 engages with the channel or notches 126 at the corner edges of an adjacent lancet body 112. It is to be understood that although a groove 126 is shown extending the length of the corner edge between the third side 124 and the two opposing sides 120, 122, the groove 126 can also be configured to extend only a portion of the corner edges for receiving a complementary shaped groove or leg 128 from an adjacent lancet body 112. By connecting adjacent lancet bodies 112 together, multiple lancet bodies 112 can engage with one another to form a stack (FIG. 3).

The material of the lancet body 112 has a resiliency such that when the first and second opposing sides 120, 122 of an adjacent lancet body 112 are connected to an adjacent lancet body 112, the channel or leg 128 grips the corresponding channel or notch 126 such that a force is required to disconnect one lancet body 112 from an adjacent lancet body 112.

As seen in FIG. 3, the U-shaped configuration of the lancet body 112 provides a gap 130 between adjacent connected lancets 110. The gap 130 provides a finger grip for removing an individual lancet 110 from the stack. A cap 132 covers the sharp tip 114 to prevent inadvertent contact with a person's skin during the connection and removal of an individual lancet 110 to and from the stack while also maintaining sterility of the sharp tip 114.

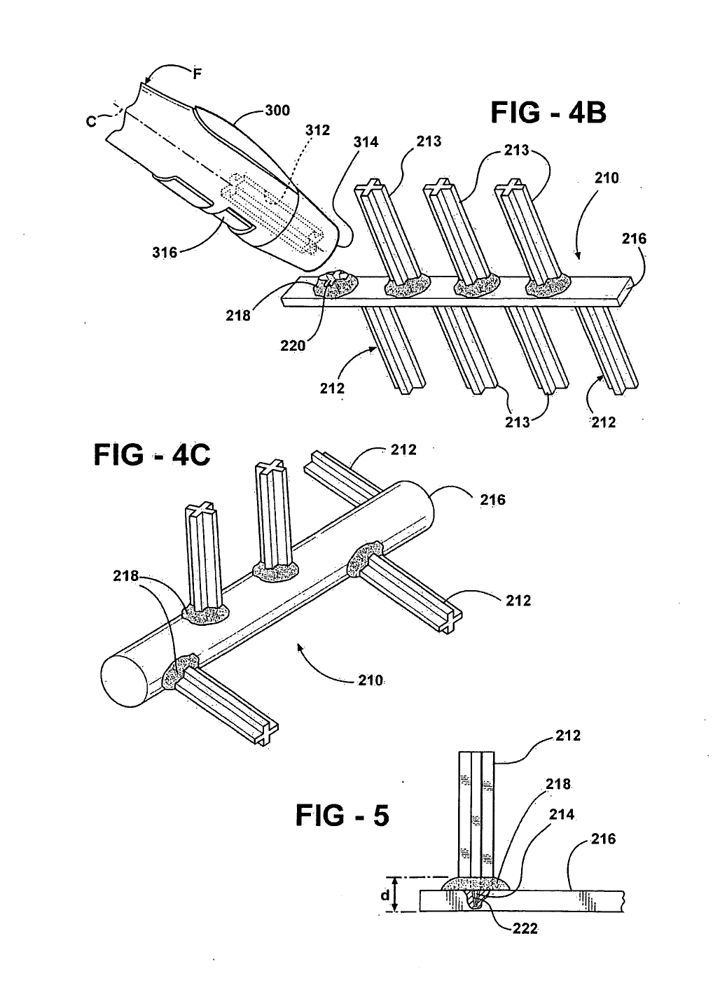

Referring to FIGS. 4A through 4G, a lancet package 210 is provided defining a carrier for holding a plurality of lancets 212. The lancet package 210 includes a plurality of lancets 212, each having a body 213 terminating at a sharp tip 214. The carrier includes an elongated member 216 frangibly attached to each of a plurality of lancets 212. The frangible connection 218 is configured to cover the sharp tip 214 of each of the plurality of lancets 212 in the form of a cap 218 as shown in FIG. 5.

In the illustrated embodiment, the body 213 of the lancet 212 has an elongate configuration configured to facilitate gripping and removal of the lancet 212 by a person's fingers or by a lancing device 300. In the embodiment shown in FIGS. 4A through 5, the base; has an X-shaped cross sectional configuration. However, the cross sectional configuration of the body 213 can include other patterns.

The plurality of lancets 212 are attached to the elongated member 216 by frangible connections 218 defined as caps. The elongated member 216 defines a sterility strip by maintaining the sharp tip 214 in a sterile environment. The caps 218 can be integrally molded to the elongated member 216 or otherwise attached in a spaced relationship along the length of the elongated member 216. The configuration of an elongated member 216 to carry a plurality of lancets also minimizes the manufacturing process.

The elongated member 216 can be configured as a planar strip as shown in FIGS. 4A and 4B or in a tubular configuration as shown in FIG. 4C. Other configurations for the elongated member 216 are envisioned provided they have a depth to accommodate a plurality of lancets 212. As can be seen in FIG. 5, the depth of the elongated member 216 along with the depth (d) of the cap 218 is configured to receive and maintain a sharp tip end 214 and body 213 in a secure and sterile manner.

The cap 218 has an opening 220 for receiving the sharp tip 214 and the adjacent portion of the body 213 of the lancet 212. The opening 220 is configured and shaped to accommodate and receive the portion of the body 213. The opening 220 provides access to a cavity 222 in the elongated member 216 for receiving the sharp tip 214. Since the cavity 222 extends a portion through the depth of the elongated member 216, is advantageous to align the lancets 212 along one side of the elongated member 216 as shown in FIG. 4A, or align the lancets 212 along opposing sides of the elongated member as shown in FIG. 4B wherein one side of the lancets 212 is offset from the opposing sides of lancets 212. Another arrangement for positioning the lancets is shown in FIG. 4C where the lancets 212 are staggered along the length of the elongated member 216 at various angles from each other so that no two, lancets are directed toward each other. It is further understood that in cases where the sharp tip 214 is very small, the cavity 222 is not required in the elongated member 216 and the sharp tip 214 of the lancet 212 terminates within the cap 218.

The lancets 212 are frictionally held within the caps 218 so that they can be easily snapped away from the elongated member 216 and also snapped back into the caps 218 after use for later disposal when all of the lancets have been used. The lancet 212 can be manually removed from the elongated member 216 by means of a person's fingers for manually loading into a lancing device 300. As an alternative, a lancing device 300 can be configured to grasp, the lancet body 213 and remove it from the sterility strip 216.

In a simplified explanation, a lancing device 300 includes an elongated housing 310 with an elongated interior chamber 312 therein for holding a lancet 212. The chamber 312 is open at a skin-contact end 314 of the lancing device 300. The lancing device 300 also includes a lancet engaging mechanism 316 within the housing 310 positioned for engagement to the lancet 212.

The lancet 212 is loaded into the lancing device 300 by placing the skin-contact end 314 of the lancing device 300 over one of the bodies 213 of the lancets 212 such that the lancet is encompassed within the chamber 312 and engaged to the lancet engaging mechanism 316. A force (F) to the lancing device 300 in a perpendicular direction to a central axis (C) is sufficient to detach the lancet 212 from the frangible connection 218 and the elongated member 216. The lancet engaging mechanism 216 can then be manually armed to move the lancet 212 so that the sharp tip 214 is held within the chamber 312 of the lancing device 300. The lancing device 300 is then in condition for firing to draw blood from a person's fingertip or other body part.

While the invention has been described in connection with what is presently considered to be the most practical and preferred embodiment, it is to be; understood that the invention is not to be limited to the disclosed embodiments but, on the contrary, is intended to cover Various modifications and equivalent arrangements included within the spirit and scope of the appended claims, which scope is to be accorded the broadest interpretation so as to encompass all such modifications and equivalent structures as is permitted under the law.

What is claimed is:

1. A lancet comprising:
a lancet body having opposing ends, a first opposing side, and a second opposing side, said first opposing side and second opposing side defined by surfaces disposed between the opposing ends; and
a sharp tip protruding from one of the opposing ends, wherein the first and second opposing sides of the lancet body are configured to fittingly engage at least one of a first and second opposing side of art adjacent lancet body.

2. The lancet of claim 1 further comprising a cap configured to cover the sharp tip and frangibly attached to the lancet body.

3. The lancet of claim 1, wherein the first opposing side of the lancet has a projection and the second opposing side of the lancet has a depression, the projection and depression configured so that the depression of the lancet body securely engages a projection of the adjacent lancet body.

4. The lancet of claim 3, wherein the projection and depression, are configured to prevent misalignment of the lancet body and the adjacent lancet body when the depression of the lancet body is engaging the projection of the adjacent lancet body.

5. The lancet of claim 3, wherein the lancet body is configured in a cubic formation.

6. The lancet of claim 1 further comprising a third side, wherein the first and second opposing sides and the third side form a U-shaped configuration.

7. The lancet of claim 6, wherein each of the first and second opposing sides, is configured with a channel formed, inside an edge spaced from the third side, the channels of the lancet body engaging with edges of the adjacent lancet body, and wherein the lancet body engaged with the one or more adjacent lancet body forms a stack.

8. The lancet of claim 1, wherein the first and second opposing sides of the lancet body connect to the at least one of the first and second opposing sides of the adjacent lancet body such that force is required to disconnect the lancet body from the adjacent lancet body.

9. A lancet package comprising:
a plurality of lancets each terminating in a sharp tip; and
an elongated member frangibly attached to each of the plurality of lancets and configured to cover the sharp tip of each of the plurality of lancets in the manner of a cap.

10. The lancet package of claim 9, wherein the elongated, member is a sterility strip.

11. The lancet package of claim 10, wherein the strip is configured with a depth sufficient to accommodate the plurality of lancets on opposite sides of the sterility strip.

12. The lancet package of claim 11, wherein the plurality of lancets are configured in an offset arrangement on the opposite sides to the sterility strip.

13. The lancet package of claim 9, wherein the plurality of lancets are configured on the elongated member, to accommodate placement of an open end of a lancing device around one of the plurality of lancets such that the lancet is detached from the elongated member when sufficient force is applied to the lancing device, thereby loading the lancet into the lancing device.

14. The lancet package of claim 9, wherein the elongated member is disposable.

15. A method of loading a lancing device having a skin contact end with a piercing aperture comprising:
providing a lancet package comprised of a plurality of lancets each terminating in a sharp tip and an elongated member frangibly attached to each of the plurality of lancets and configured to cover the sharp; tip of each of the plurality of lancets in the manner of a cap;
placing the skin contact end of the lancing device over one of the plurality of lancets such the lancet engages a lancet engaging mechanism in the lancing device;
applying a force perpendicular to a central axis of the lancing device sufficient to detach the lancet from the elongated member; and
arming the lancing device by actuating the lancet engaging mechanism to move the lancet so that the sharp tip is within the lancing device.

16. The method of claim 15, wherein the elongated member is a sterility strip.

17. The method of claim 16, wherein the sterility strip is configured with a depth sufficient, to accommodate the plurality of lancets on opposite sides of the sterility strip.

18. The method of claim 17, wherein the plurality of lancets are configured in an offset arrangement on the opposite sides to the sterility strip.
